(19) United States
(12) Patent Application Publication
(10) Pub. No.: US 2001/0049586 A1
(43) Pub. Date: Dec. 6, 2001

(54) Iterative analysis of non-responding population in the design of pharmacogenetic studies

(76) Inventor: Allen David Roses, Durham, NC (US)

Correspondence Address: DAVID J LEVY, VP INTELLECTUAL PROPERTY, GLAXO WELLCOME INC, GLOBAL INTELLECTUAL PROPERTY, FIVE MOORE DR, PO BOX 13398, RESEARCH TRIANGLE PARK, NC, 27709-3398, US

(21) Appl. No.: 09/826,629

(22) Filed: Apr. 5, 2001

Related U.S. Application Data
(60) Provisional application No. 60/194,789, filed on Apr. 5, 2000.

(51) Int. Cl. G01N 33/48; G01N 31/00; G01N 37/00; A01N 43/04; A01N 61/00

(52) U.S. Cl. 702/19; 514/2; 514/44; 702/22; 702/82; 702/83

(57) ABSTRACT

A method of designing human clinical drug trials in which the drug under study is administered to a defined population of test patients who have demonstrated a predisposition to responding to the study drug by virtue of their having one or more genes that have independently been determined to be associated with the desired response.

### CROSS-REFERENCE TO RELATED APPLICATION

This application claims the benefit of and priority to application number 60/194,789 filed Apr. 5, 2000, which status is pending and the entirety of which is incorporated herein by reference.

### FIELD OF THE INVENTION

The present invention relates to methodology in the design and conduct of human clinical drug trials. It particularly relates to such methods by use of pharmacogenetics data.

### BACKGROUND OF THE INVENTION

Presently, the marketing of novel medicines requires extensive clinical trials conducted to demonstrate the efficacy and safety of the candidate medicine. In any given clinical trial, there will be an observed percentage of the patients enrolled in that trial who will either respond to the candidate medicine as hoped for, or who will respond less strongly, or who will not respond at all. Also, there are three possible adverse side effect outcomes, namely no adverse side effect, some acceptable degree of adverse side effect, and an unacceptable adverse side effect. Currently available data suggest that a major part of the partial responders or non-responders populations results from multiple etiologies leading to the recognized phenotype. Single nucleotide polymorphism (SNP) profiling of different medicine-responsive association groups during such clinical studies implies that the location of genes contributing to heterogeneous forms of the disease can also be identified, leading to the discovery of additional susceptibility targets.

A systematic study of those populations of patients who respond to a given medicine is complemented by a study of the converse, that is, a systematic study of those subgroups of patients who did not respond with efficacy and acceptable safety outcomes to the initially studied medicine. Such partial responders or non-responders could be identified in real time, rather than by the current trial and error system that performs this function once a medicine appears in the marketplace. Currently drugs are broadly marketed to many patients who will not benefit and who may experience adverse reaction. Little information of use is obtained from these patients currently, other than broad warnings for use in product labeling.

There is thus a need in the art for a method of designing human clinical drug trials in a fashion that benefits available patient populations through 1) minimizing the likelihood of adverse events, 2) maximizing the likelihood of therapeutic response and 3) providing a pool of data that readily suggest a subsequent clinical trial that is capable of utilizing all prior data, whether for responders or for non-responders+.

### SUMMARY OF THE INVENTION

In the brief summary, the invention is a method of conducting clinical drug trials by pharmacogenetic stratification of a patient population, comprising the steps of conducting a first clinical drug trial on a patient population, such that said drug trial identifies an association between a phenotype and a genotype (a); separating said patient population in said clinical drug trial into sub-populations of responders and non-responders (b); conducting a subsequent clinical drug trial on a non-responder patient population such that said subsequent drug trial identifies a subsequent association between a phenotype and genotype (c); separating the patient population of step (c) into subsequent responder and subsequent non-responder patient populations (d); and then repeating steps (c)and (d) through as many iterations as desired.

Clinical trials o the type discussed in this application generate various kinds of data that is advantageously stored on electronically readable media, including, but not limited to magnetic tapes, magnetic disks, solid sate memory and storage devices, optically readable disks and any combination of these. Such data is also advantageously transmitted or communicated via telecommunications means including metallic or optical fiber lines or via wireless electromagnetic frequency devices. Additionally, such data is advantageously communicated via at least two or more electronic computing devices, including personal computers, computer workstations, computer servers, mainframe computers, super computers and the like. Such communications can occur either directly from device to device or through a plurality of such devices that have been electronically instructed on how to route such communications from a sender to a designated receiver of such communication. Such data that can be stored and communicated in the above ways include, but are not limited to, any nucleotide sequence data, amino acid sequence data, protein-protein interaction data, clinical diagnosis data or statistics data generated by the above clinical trials. The use of such electronic devices as described is an alternative embodiment of the invention claimed herein, particularly when used commercially.

### BRIEF DESCRIPTION OF THE FIGURES

The following Figures are used to help illustrate this invention.

FIG. 1 is a graph based on data collected from female patients enrolled in clinical trials of alosetron for the treatment of IBS. The study population was comprised of non-constipated individuals with IBS. The subjects were divided into 5HTT genotypes. Of the 219 subjects, 71(32.4%) were genotype del/del (deletion/deletion, alternatively denoted as “1/1”) 5HTT, 75 (34.2%) were genotype del/ins (deletion/insertion, alternatively denoted as “1/2”) 5HTT and 73 (33.3%) were genotype ins/ins (insert/insert, alternatively denoted as “2/2”) 5HTT. FIG. 1 shows the percentage of subjects that responded to treatment with either alosetron or a placebo, divided into 5HTT genotypes. “Response” was defined as relief of IBS symptoms (abdominal pain and discomfort) during six weeks of a twelve week treatment study. Two hundred and nineteen subjects, received either alosetron (102 subjects) or matched placebo (117 subjects). The proportion of patients achieving response following treatment with alosetron was 68% (21/31) for del/del 5HTT, 64% (21/33) for del/ins 5HTT and 58% (22/38) for ins/ins 5HTT. The proportion of patients achieving response following treatment with placebo was 58% (23/40) for del/del 5HTT, 38% (16/42) for del/ins 5HTT and 34% (12/35) for ins/ins 5HTT.

FIG. 2 compares, among 5HTT genotypes, the percentage of alosetron-treated subjects who reported constipation. Of the 102 alosetron treated subjects, the proportion of subjects reporting constipation was 13% (4/31) for del/del 5HTT, 30% (10/33) for del/ins 5HTT and 21% (8/38) for ins/ins 5HTT.

FIG. 3 is a composite of the information shown in FIGS. 1 and 2, and compares among 5HTT genotypes the incidence of constipation and the percentage of subjects experiencing relief of IBS symptoms, in alosetron-treated subjects. Subjects with the del/del 5HTT genotype showed an increased incidence of favourable therapeutic response with a higher incidence of relief of IBS symptoms and a lower incidence of constipation, when compared with subjects who had del/ins or ins/ins 5HTT genotypes.

### DETAILED DESCRIPTION OF THE INVENTION

The method of the present invention is best illustrated by the following descriptions of the conduct of a pharmacogenetic study, which includes descriptions of how to conduct and interpret the various phases of the study, and the technologies underlying the conduct of the study.

Studies were conducted that relate to polymorphisms in the 5-hydroxytryptamine transporter (5-HTT) gene, and phenotypes that are associated or correlated therewith. More particularly, the studies related to the correlation of such polymorphisms to the response of subjects with gastrointestinal disorders (such as Irritable Bowel Syndrome (IBS)) to pharmaceutical treatment. Such studies further related to methods of screening compounds for pharmaceutical activity. The present studies also relate to methods of genotyping subjects for predictive purposes, again, based upon said correlations.

Many gastrointestinal disorders of unknown etiology, including Irritable Bowel Syndrome (IBS), are believed to be multifactorial disorders. In many of these disorders, no biochemical marker has been found and diagnosis is accomplished primarily by observation of clinical symptoms. Unlike single gene Mendelian disorders, complex disorders such as diabetes, migraine and cardiovascular disease tend to be multifactorial and are caused by the interaction of one or more susceptibility genes with environmental factors. To date, no individual susceptibility genes for IBS have been identified by either linkage or association studies.

Irritable Bowel Syndrome (IBS) is a common gastrointestinal disorder characterized by abdominal pain and discomfort, and altered bowel habit. IBS may be characterized by symptoms of either constipation or diarrhea, or alternating constipation and diarrhea. Currently, there are no single pathophysiological or diagnostic markers of IBS. However, various diagnostic criteria for IBS are available, e.g., Thompson et al., Gastroent. Int. 2:92 (1989); Manning et al., Br. Med. J. 2:653 (1978); Thompson et al., Gut 45:1143 (1999)

Antagonism at 5-hydroxytryptamine receptors, such as by alosetron hydrochloride, has been shown to be useful in the treatment of diarrhea-predominant irritable bowel syndrome.

Alosetron hydrochloride (CAS registry number: CAS-122852-69-1; see U.S. Pat. No. 5,360,800, the entire disclosure of which is incorporated herein by reference) is a 5-HT3 receptor antagonist. Both animal and human studies indicate that 5-HT3 receptor blockade has therapeutic value in the treatment of irritable bowel syndrome, particularly in diarrhea-predominant IBS. (The disclosures of all US patents cited herein are incorporated herein by reference in their entirety.)

In double blind, placebo controlled studies, alosetron hydrochloride has been shown to reduce pain and improve bowel function in patients with Irritable Bowel Syndrome (IBS). See Bardhan et al., Aliment Pharmacol Ther 2000 Jan;14(1):23-34: Jones et al., Aliment Pharmacol Ther 1999 Nov;13(11):1149-27; Camilleri et al., Ailment Pharmacol Ther 1999 (Sept; 13(9):1149-59, Mangel et al., Aliment Pharmacol Ther 1999 May;13 Suppl 2:77-82. Alosetron has further been indicated as a potential treatment for the symptomatic relief of carcinoid diarrhea. Saslow et al., Gut 1998 May;42(5):628-34.

5-hydroxytryptamine (5HT) receptors have been identified and characterized in the gastrointestinal tract, including 5HT3, 5HT4, and 5HT1a receptors; these receptors are involved not only in modulating gut motility but also in visceral sensory pathways. Various 5HT3 antagonists (e.g., alosetron, granisetron and ondansetron) have been identified for the treatment of IBS. This class of drug appears to reduce visceral sensitivity and have inhibitory effects on motor activity in the distal intestine. Full and partial 5HT4 agonists (e.g., HTF919, tegaserod) are potential therapeutics to improve constipation-predomninant IBS. Farthing et al., Baillieres Best Pract Res Clin Gastroenterol. 1999 Oct;13(3):461-71. 5HT4 antagonists (piboserod, SB-207266A) have also been suggested for the treatment of IBS.

The human 5HTT protein is encoded by a single gene (SLC6A4) found on chromosome 17q12 (Ramamoorthy et al., Proc. Natl. Acad. Sci. USA 90:2542 (1993); Gelemter et al., Hum. Genet. 95:677 (1995); Lesch et al., J. Neural Transm. 91:67 (1993)). The 5HT Transporter regulates the magnitude and duration of serotonergic responses. An insertion/deletion polymorphism consisting of a 44 base pair segment in the transcriptional control region 5′ upstream to the 5HTT coding sequence has previously been identified. The deletion (or short) allele of this polymorphism is associated with decreased transcription efficiency of the 5HTT gene promoter, decreased gene expression, and decreased 5-hydroxytryptamine uptake. (Heils et al., J. Neural Transm. 102:247 (1995); Heils et al., J. Neurochem 66:2621 (1996), Lesch et al., Science 274:1527 (1996)). Additionally, various biochemical studies suggest that 5HT uptake function is frequently reduced in psychiatric illnesses, and variation in functional 5HTT expression due to 5HTT promoter polymorphism has been implicated as a potential genetic susceptibility factor for affective disorders (Collier et al., Mol Psychiatry 1996 Dec;1(6):453-60; Lesch et al., Science 1996 Nov 29;274(5292):1527-31; Furlong et al., Am J Med Genet 1998 Feb 7;81(1):58-63; Menza et al., J Geriatr Psychiatry Neurol 1999 Summer;12(2):49-52; Rosenthal et al., Mol Psychiatry 1998 Mar;3(2):175-7).

It has been determined that polymorphisms in the 5-hydroxytryptamine transporter (5HTT) gene are correlated with the response of subjects with IBS to pharmaceutical therapy (Applicants co-pending application, PCT/USO1/04755, the entire disclosure of which is incorporated herein by reference. More particularly, it was found that an insertion/deletion polymorphism in the 5′ non-coding region of the 5HTT gene is a predictor for the response of patients with IBS to treatment with a 5HT antagonist; and there was identified a genetic subset of IBS patients that displays a higher incidence of relief of IBS symptoms and a lower incidence of the side effect of constipation when treated with alosetron (compared to patients with an alternative polymorphism at the same site of the 5HTT gene).

Consequently, these observations led to a method of screening a patient population to identify those subjects with an increased likelihood of responding favorably to a treatment with a 5HT antagonist for a gastrointestinal disorder. The subjects may have been previously diagnosed as having IBS, or the screening may be used in conjunction with IBS diagnostic efforts.

A further aspect is a method of screening a subject suffering from a gastrointestinal disease that is treatable with a 5-hydroxytryptamine (5HT) ligand, as an aid in predicting the subject's response to treatment with a 5HT ligand. The method compromises obtaining a sample of the subject's DNA and determining the genotype of the subject at a polymorphic allelic site in the 5hydroxytryptamine transporter (5HTT) gene, where different genotypes at that site have been associated with different incidences of a phenotypic response to treatment with a 5HT ligand. The genotype that is detected in the sample indicates that the subject is likely to have the phenotypic response associated with that genotype.

Another aspect is a method of screening a subject with irritable bowel syndrome (IBS), as an aid in predicting the subject's response to treatment with a 5HT ligand. The method compromises obtaining a sample of the subject's DNA and determining the genotype of the subject at a polymorphic allelic site in the 5hydroxytryptamine transporter (5HTT) gene, where different genotypes at that site have been associated with different incidences of a phenotypic response to treatment with a 5HT ligand.

A further aspect is a method of screening a 5-hydroxytryptamine (5HT) ligand for variations in a measurable phenotypic effects among genetic subpopulations of subjects with a gastrointestinal disorder. The method compromises administering the 5HT ligand to a population of subjects suffering from the gastrointestinal disorder, and obtaining DNA samples from each of the subjects. The DNA samples are genotypes for a polymorphic allele of the 5hydroxytryptamine transporter (5HTT) gene, and correlations between the polymorphic allele genotype and the occurrence of a phenotypic response in the population of subjects are determined. Detection of a genotype that is correlated with an increased or decreased incidence of a desired therapeutic response or a side effect (compared to the incidence in subjects with alternative genotypes) indicates that the effectiveness of the ligand in treating that gastrointestinal disorder varies among genetic subpopulations.

Genetic samples were obtained from subjects enrolled in clinical trials of alosetron for the treatment of IBS. The genetic samples were screened for an insertion/deletion polymorphism in the 5′ non-coding region of the 5-hydroxytryptamine transporter gene (5HTT gene), using polymerase chain reaction (PCR) technology. The alleles were labeled as “del” (deletion) or “ins” (insertion) resulting in three possible genotypes (del/del; del/ins or ins/ins). The insertion polymorphism (allele “ins”) had SEQ ID NO:2:

| | |
|---|---|
| ggcgttgccg ctctgaatgc cagccctaac ccctaatgtc cctactgcag cctcccagca 60 | (SEQ ID NO:2) |
| tcccccctgc aacctcccag caactccctg tacccctcct aggatcgctc ctgcatcccc 120 | |
| cattatcccc cccttcactc ctcgcggcat cccccctgca ccccccagca tcccccctgc 180 | |
| agccccccca gcatctcccc tgcaccccca gcatcccccc tgcagccctt ccagcatccc 240 | |
| cctgcacctc tcccaggatc tcccctgcaa cccccattat cccccctgca cccctcgcag 300 | |
| tatcccccct gcacccccca gcatcccccc atgcaccccc ggcatccccc ctgcacccct 360 | |
| ccagcattct ccttgcaccc taccagtatt cccccgcatc ccggcctcca agcctcccgc 420 | |
| ccaccttgcg gtccccgccc tggcgtctag gtggcaccag aatcccgcgc ggactccacc 480 | |
| cgctgggagc tgccctcgct tgcccgtggt tgtccagctc agtc cctc | |

Legend: PCR primer sequences are in underlined typeface

Non-coding sequences are shown in lowercase typeface

Polymorphic bases are shown in bold typeface

Base numbering is relative to the sequence shown

Polymorphism numbering is relative to the gene cDNA sequences

The “del” allele represents a deletion of approximately 44 base pairs in the 5′ untranslated region of the 5HTT gene. This deletion in the transcriptional regulatory region has been associated with decreased re-uptake of 5HT and therefore an increased 5HT basal level. Therefore, the del/del genotype is postulated to result in a lower transcription efficiency, lower production of 5HTT, and reduced basal 5HT re-uptake (compared to the del/ins or ins/ins genotype). The del/del, del/ins and ins/ins genotypes were approximately evenly distributed among the subjects. Of 219 subjects, 71 were del/del 5HTT; 75 were del/ins 5HTT; and 73 were ins/ins 5HTT.

It was further determined that the del/del genotype is associated with an increased incidence of relief of IBS symptoms and a lower frequency of constipation as an effect of treatment with a 5HT3 antagonist, and therefore an increased incidence of favorable therapeutic response to treatment with a 5HT3 antagonist (compared to subjects with the del/ins or ins/ins genotype treated with the same 5HT3 antagonist).

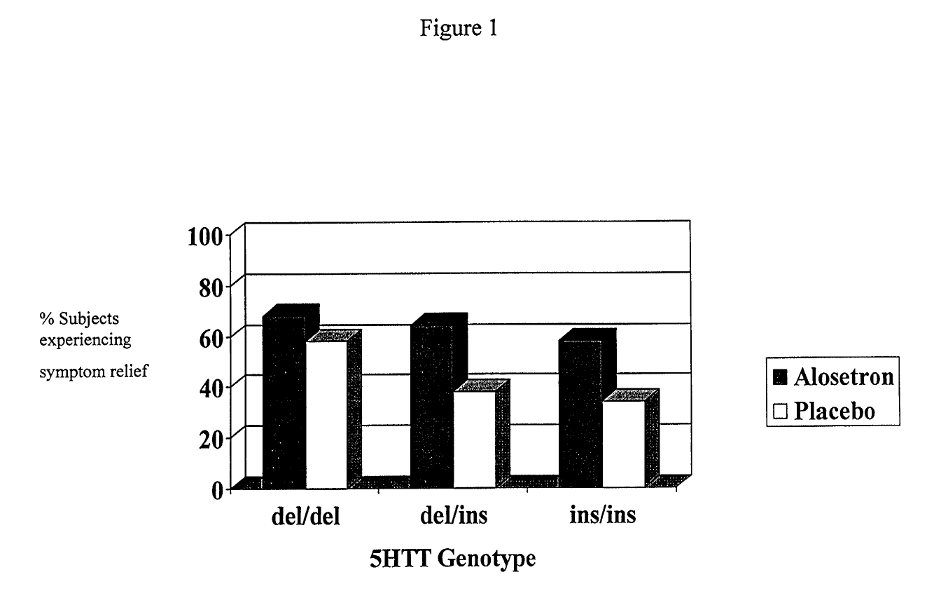

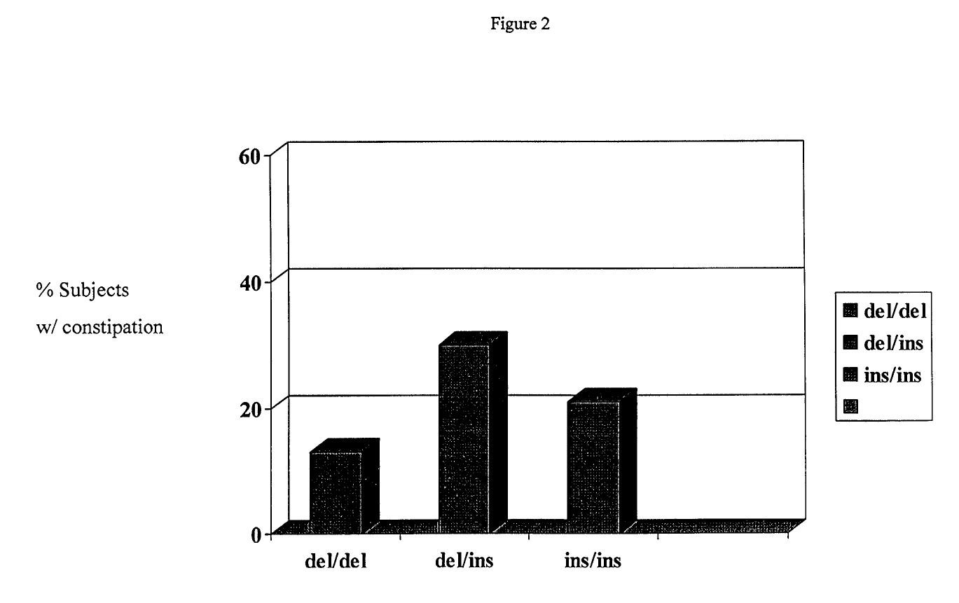

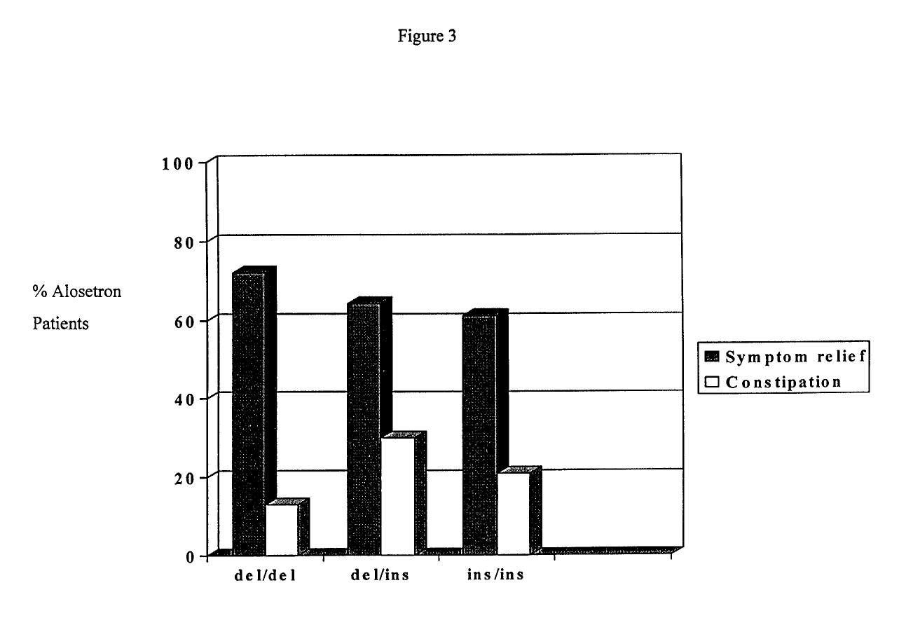

In each of the three 5HTT genotypes alosetron was more effective than placebo in relieving IBS symptoms (FIG. 1). However, in the del/del genotype group (homozygous for the deletion polymorphism), the incidence of relief of IBS symptoms for both alosetron and placebo was increased compared to other 5HTT genotypes (FIG. 1). Subjects with the del/del genotype also showed a reduced incidence of constipation compared to the del/ins and ins/ins 5HTT genotype groups (FIG. 2). FIG. 3 shows subjects with the del/del 5HTT genotype showed an increased incidence of favourable therapeutic response with a higher incidence of relief of IBS symptoms and a lower incidence of the alosetron-induced side effect of constipation, when compared with subjects who had del/ins or ins/ins 5HTT genotypes.

Accordingly, a subject who suffers from a gastrointestinal disease that is treatable with 5HT ligands can be genetically screened, to aid in predicting their response to such treatment. Screening comprises obtaining a sample of DNA from the subject and screening the DNA to determine the genotype (presence/absence of polymorphic alleles) at a predetermined polymorphic site in the 5hydroxytryptamine transporter (5HTT) gene, where different genotypes at that site have previously been associated with different incidences of a phenotypic response to treatment with a 5HT ligand. The presence of a particular genotype therefore indicates an increased likelihood that the individual subject will exhibit the associated phenotype. The genotype will rarely be absolutely predictive, i.e., where a population with a certain genotype displays a high incidence of a particular phenotype, not every individual with that genotype will display the phenotype. However, it will be apparent to those skilled in the art that genotyping a subject as described herein will be an aid in predicting the response a subject will have to treatment with a 5HT ligand, and thus assist in the treatment decision.

As used herein, “genotyping a subject (or DNA sample) for a polymorphic allele at a defined genomic locus” or “determining the genotype at a polymorphic allelic site” means detecting which forms of the allele are present in a subject (or a sample). As is well known in the art, an individual may be heterozygous or homozygous for a particular allele. More than two forms of an allele may exist, as is the case with microsatellite markers; thus there may be more than three possible genotypes.

As used herein, a subject that is “predisposed to” a particular phenotypic response based on genotyping of a polymorphic allele will be more likely to display that phenotype than an individual with a different genotype at that polymorphic allele. Where the phenotypic response is based on a biallelic polymorphism, the response may differ among the three possible genotypes (Eg. For 5HTT: del/del, del/ins and ins/ins).

As used herein, a “genetic subset” of a population consists of those members of the population having a particular genotype. In the case of a biallelic polymorphism, a population can potentially be divided into three subsets: homozygous for allele 1, heterozygous, and homozygous for allele 2.

As used herein, a gastrointestinal disease ‘treatable with 5HT ligands’ is one in which the administration of a 5HT ligand (in an appropriate pharmaceutical formulation, and in a therapeutically effective amount) has been shown to reduce or alleviate symptoms, without causing unacceptable side effects. Such therapeutic effectiveness is typically evidenced by Regulatory Authority (eg FDA, EMEA)approval of the pharmaceutical preparation, or by publication of the results of clinical studies in peer-reviewed medical journals. Therapeutically effective amounts of such compounds can be readily determined by those skilled in the art using, e.g., dose-response studies. As used herein, the term ‘5HT ligand’ encompasses antagonists and agonists of 5HT receptors, including partial agonists and drugs that interact with 5HTT (eg selective serotonin re-uptake inhibitors, SSRI's). 5HT ligands may bind to any subtype of the 5HT receptor, including 5HT3 and 5HT4 receptors; the ligands may be specific for a particular receptor subtype.

Known 5HT-related compounds include 5HT3 antagonists (e.g., ondansetron, granisetron, tropisetron, dolasetron, mirtazapine, itasetron, pancopride, zatosetron, azasetron, cliansetron, YM-144 (Yamanouchi) and RS17017 (Roche)).

5HT4 agonists are also known, including tegaserod, prucalopride, norcisapride and the 4-amino-5-chloro-2-methoxy-N-(1-substituted piperidin-4-yl)benzamide known as Y-34959 (Yoshitomi Pharmaceuticals), and buspirone. The use of 5HT4 agonists to treat constipation-predominant IBS has been proposed. 5HT4 antagonists include piboserod (SmithKline Beecham).

Dual 5HT3 and 5HT4 agonists include renzapride (SmithKline Beecham) and E3620 (Eisai). A 5HT1a agonist is also known, LY315535 (Eli Lilly).

Selective serotonin re-uptake inhibitors include fluoxetine, etc.

As used herein, a “side effect” is an undesirable response to the administration of a therapeutic compound, i.e., an effect that is not directed to alleviating the symptoms or cause of the disease being treated. Side effects range from minor inconveniences to more serious events.

According to the present methods, a compound with 5HT ligand activity may be screened for variation in its effects among genetic subpopulations of subjects with a gastrointestinal disorder. Such methods involve administering the compound to a population of subjects suffering from a 5HT-mediated gastrointestinal disorder, obtaining DNA samples from the subjects (which may be done either prior to or after administration of the compound), genotyping a polymorphic allelic site in the 5HTT gene, and correlating the genotype of the subjects with their phenotypic responses (both favorable and unfavorable) to the treatment. A genotype that is correlated with an increased incidence of a desired therapeutic response (or a decreased incidence of an undesirable side effect), compared to the incidence in subjects with alternative genotypes at the polymorphic allelic site, indicates that the effectiveness of the compound in treating such gastrointestinal disorder varies among genetic subpopulations.

Stated another way, the method may be used to determine the correlation of a known 5HTT polymorphic allele with the response of subjects with gastrointestinal disorders (such as IBS) to treatment with a 5HT ligand. The population of subjects with the disease of interest is stratified according to genotype for the particular polymorphic allele, and their response to a therapeutic agent is assessed (either prospectively or retrospectively) and compared among the genotypes. The response to the therapeutic agent may include either, or both, desired therapeutic responses (e.g., the alleviation of signs or symptoms) and undesirable side effects. In this way, genotypes that are associated with an increased (or decreased) incidence of therapeutic efficacy, or an increased (or decreased) incidence of a particular side effect, may be identified. The increase or decrease in response is in comparison to the other genotypes, or to a population as a whole. Once this relative increase or decrease has been observed, responders and non-responders can be identified and assigned to separate sub-populations. A non-responder will be a subject displaying a defined degree of decreased incidence of therapeutic efficacy, possibly displaying no therapeutic efficacy at all. Alternatively, a non-responder can be categorized as a subject displaying a defined degree of increased incidence of a side effect of interest ranging from relatively benign side effects to those that are potentially life-threatening.

Polymorphisms are variant sequences within the human genome that may or may not have a functional consequence. These variants can be used in all aspects of genetic investigation including the analysis and diagnosis of genetic disease, forensics, evolutionary and population studies. Two types of genetic analyses are typically performed: linkage and association studies.

A linkage study provides genetic map information where there is no prior knowledge or assumption about the function of a gene. In a linkage study one uses DNA polymorphisms to identify chromosomal regions that are identical between affected relatives with the expectation that allele sharing frequencies will be higher for a marker (polymorphism) whose chromosomal location is close to that of the disease allele. Physical cloning of a linkage region narrows down the DNA sequence that could harbor the candidate disease gene. While linkage analysis locates the disease locus to a specific chromosome or chromosome region, the region of DNA in which to search for the gene is typically large, on the order of several million base pairs.

In contrast to linkage, association shows the coexistence of a polymorphism and a disease phenotype in a population. Association studies are based upon linkage disequilibrium, a phenomenon that occurs between a marker and a disease phenotype if the marker polymorphism is situated in close proximity to the functional (disease) causing variant. Since the marker and disease causing variant are in close proximity, it requires many generations of recombination to separate them in a population. Thus they tend to co-exist together on the same chromosome at a higher than expected frequency. A marker (polymorphism) is said to be associated with a specific phenotype when its frequency is significantly higher among one phenotype group compared to its frequency in another. In general, the closer a marker is to the functionally polymorphic site, the stronger the association. Association studies offer the opportunity to finely map linkage regions, map loci refractory to linkage analysis and map unknown predisposition loci. Polymorphisms that are in linkage disequilibrium with each other can be spaced over large regions. Linkage disequilibrium has been reported in regions as small as 1kb or as large as 500 kb. Polymorphisms throughout a gene can be in linkage disequilibrium with each other, such that it is valuable to study the whole genome structure—introns, exons, promoters and transcriptional regulatory regions, and 3′ and 5′ untranslated regions. A marker that is in linkage disequilibrium with a functional polymorphism can be used as the basis of a test that correlates that polymorphism with a phenotype of interest.

A polymorphism in the 5HTT gene plays a role in the response of subjects to pharmaceutical treatment of IBS, thus the genotyping of the 5HT Transporter (5HTT) gene (either directly or via its expression product) will be useful in identifying therapeutic compounds with measurable effects that vary among 5HTT genotypes. The effect to be measured will depend on the particular gastrointestinal condition, therapeutic compound, and patient population, as will be apparent to one skilled in the art. The measurable effect may be the relief of, or change in, a pathologic sign or symptom or the occurrence of a side effect related to compound administration. Measurement may be objective or subjective (e.g., by patient self-reporting). The association of a 5HTT genotype with a therapeutic response will provide a method of determining the probability that an individual subject will respond in a particular way to treatment with 5HT ligands. In genotyping, the characteristic that is typically measured is one that can be influenced by a polymorphism in the gene or its expression product. As used herein, the term polymorphism includes Single Nucleotide Polymorphisms (SNPs), insertion/deletion polymorphisms; microsatellite polymorphisms; and variable number of tandem repeat (VNTR) polymorphisms.

Methodologies in the Detection of Polymorphisms

Polymorphic alleles are typically detected by directly determining the presence of the polymorphic sequence in a polynucleotide or protein from the subject, using any suitable technique that is known to those of ordinary skill in the art. Such a polynucleotide is typically genomic DNA, or a polynucleotide derived from this polynucleotide, such as in a library made using genomic material from the individual (e.g. a cDNA library). The processing of the polynucleotide or protein before the carrying out of the method of the invention is further discussed below. Typically the presence of the polymorphism is determined in a method that comprises contacting a polynucleotide or protein of the individual with a specific binding agent for the polymorphism and determining whether the agent binds to the polynucleotide or protein, where the binding indicates that the polymorphism is present. The binding agent may also bind to flanking nucleotides and amino acids on one or both sides of the polymorphism, for example at least 2, 5, 10, 15 or more flanking nucleotide or amino acids in total or on each side. In one embodiment the agent is able to bind the corresponding wild-type sequence by binding the nucleotides or amino acids which flank the polymorphism position, although the manner of binding will be different than the binding of a polymorphic polynucleotide or protein, and this difference will be detectable (for example this may occur in sequence specific PCR as discussed below).

In the case where the presence of the polymorphism is being determined in a polynucleotide it may be detected in the double stranded form, but is typically detected in the single stranded form.

The binding agent may be a polynucleotide (single or double stranded) typically with a length of at least 10 nucleotides, for example at least 15, 20, 30, or more polynucleotides. The agent may be a molecule that is structurally similar polynucleotides, comprising units (such as purines or pyrimidines) able to participate in Watson-Crick base pairing. The agent may be a protein, typically with a length of at least 10 amino acids, such as at least 20, 30, 50, 100 amino acids. The agent may be an antibody (including a fragment of such an antibody that is capable of binding the polymorphism).

A polynucleotide agent which is used in the method will generally bind to the polymorphism of interest, and the flanking sequence, in a sequence specific manner (e.g. hybridize in accordance with Watson-Crick base pairing) and thus typically has a sequence which is fully or partially complementary to the sequence of the polymorphism and flanking region.

Thus, in one method of detection, a binding agent is used as a probe. The probe may be labeled or may be capable of being labeled indirectly. The detection of the label may be used to detect the presence of the probe on (and hence bound to) the polynucleotide or protein of the individual. The binding of the probe to the polynucleotide or protein may be used to immobilize either the probe or the polynucleotide or protein (and thus to separate it from one composition or solution).

In another method of detection, the polynucleotide or protein of the individual is immobilized on a solid support and then contacted with the probe. The presence of the probe immobilized to the solid support (via its binding to the polymorphism) is then detected, either directly by detecting a label on the probe or indirectly by contacting the probe with a moiety that binds the probe. In the case of detecting a polynucleotide polymorphism the solid support is generally made of nitrocellulose or nylon. In the case of a protein polymorphism, the method may be based on an ELISA system, the techniques of which are well known to those of ordinary skill in the art.

Detection methods may be based on an oligonucleotide ligation assay in which two oligonucleotide probes are used. These probes bind to adjacent areas on the polynucleotide which contains the polymorphism, allowing (after binding) the two probes to be ligated together by an appropriate ligase enzyme. However the two probes will only bind (in a manner which allows ligation) to a polynucleotide that contains the polymorphism, and therefore the detection of the ligated product may be used to determine the presence of the polymorphism.

In another detection method, the probe is used in a heteroduplex analysis-based system to detect polymorphisms. In such a system when the probe is bound to a polynucleotide sequence containing the polymorphism it forms a heteroduplex at the site where the polymorphism occurs (i.e. it does not form a double strand structure). Such a heteroduplex structure can be detected by the use of an enzyme that is single or double strand specific. Typically the probe is an RNA probe and the enzyme used is RNAse H that cleaves the heteroduplex region, thus allowing the polymorphism to be detected by means of the detection of the cleavage products.

A detection method may be based on fluorescent chemical cleavage mismatch analysis which is described for example in PCR Methods and Applications 3:268-71 (1994) and Proc. Natl. Acad. Sci. 85:4397-4401 (1998).

In one embodiment the polynucleotide agent is able to act as a primer for a PCR reaction only if it binds a polynucleotide containing the polymorphism (i.e. a sequence- or allele-specific PCR system). Hence a PCR product will only be produced if the polymorphism is present in the polynucleotide of the individual. Thus the presence of the polymorphism may be determined by the detection of the PCR product. Preferably the region of the primer which is complementary to the polymorphism is at or near the 3′ end the primer. In one embodiment of this system the polynucleotide the agent will bind to the wild-type sequence but will not act as a primer for a PCR reaction.

Detection may be via a Restriction Fragment Length Polymorphism (RFLP) based system. This can be used if the presence of the polymorphism in the polynucleotide creates or destroys a restriction site that is recognized by a restriction enzyme. Thus treatment of a polynucleotide with such a polymorphism will lead to different products being produced compared to the corresponding wild-type sequence. Thus the detection of the presence of particular restriction digest products can be used to determine the presence of the polymorphism.

The presence of the polymorphism may alternatively be determined based on the change that the presence of the polymorphism makes to the mobility of the polynucleotide or protein during gel electrophoresis. In the case of a polynucleotide single-stranded conformation polymorphism (SSCP) analysis may be used. This measures the mobility of the single stranded polynucleotide on a denaturing gel compared to the corresponding wild-type polynucleotide, the detection of a difference in mobility indicating the presence of the polymorphism. Denaturing gradient gel electrophoresis (DGGE) is a similar system where the polynucleotide is electrophoresed through a gel with a denaturing gradient, a difference in mobility compared to the corresponding wild-type polynucleotide indicating the presence of the polymorphism.

The presence of the polymorphism may be determined using a fluorescent dye and quenching agent-based PCR assay such as the Taqman PCR detection system. In brief, this assay uses an allele specific primer comprising the sequence around, and including, the polymorphism. The specific primer is labeled with a fluorescent dye at its 5′ end, a quenching agent at its 3′ end and a 3′ phosphate group preventing the addition of nucleotides to it. Normally the fluorescence of the dye is quenched by the quenching agent present in the same primer. The allele specific primer is used in conjunction with a second primer capable of hybridizing to either allele 5′ of the polymorphism.

In the assay, when the allele comprising the polymorphism is present Taq DNA polymerase adds nucleotides to the nonspecific primer until it reaches the specific primer. It then releases polynucleotides, the fluorescent dye and quenching agent from the specific primer through its endonuclease activity. The fluorescent dye is therefore no longer in proximity to the quenching agent and fluoresces. In the presence of the allele which does not comprise the polymorphism the mismatch between the specific primer and template inhibits the endonuclease activity of Taq and the fluorescent dye in not released from the quenching agent. Therefore by measuring the fluorescence emitted the presence or absence of the polymorphism can be determined.

In another method of detecting the polymorphism a polynucleotide comprising the polymorphic region is sequenced across the region which contains the polymorphism to determine the presence of the polymorphism.

Accordingly, any of the following techniques may be utilized in the present methods for genotyping, as is known in the art.

General: DNA sequencing, sequencing by hybridization;

Scanning: PTT (Protein truncation technique), SSCP (single strand conformational analysis), DGGE (denaturing gradient gel electrophoresis), TGGE (temperature gradient gel electrophoresis), Cleavase, Heteroduplex analysis, CMC (chemical mismatch cleavage), enzymatic mismatch cleavage;

Hybridization based: solid phase hybridization (dot blots, MASDA, reverse dot blots, oligonucleotide arrays (chips)); solution phase hybridization (Taqman, Molecular Beacons);

Extension based: ARMS (Amplification Refractory Mutation System), ALEX (Amplification Refractory Mutation System Linear Extension) SBCE (Single Base Chain Extension)

Incorporation based: Mini-sequencing, APEX; (Arrayed Primer Extension)

Restriction enzyme based: RFLP (restriction fragment length polymorphism)

Ligation based: OLA (Oligonucleotide Extension Assay)

Other: Invader (Third Wave Technologies).

Test Kits

Another emobodiment of the presently claimed invention relies upon a predictive (patient care) test or test kit. This predictive test could be a product and/or a service which aids in disease management of IBS based on pre-determined associations between genotype and phenotypic response to 5HT ligands in treating gastrointestinal disease. Such a test could take two different formats:

a molecular test which analyses DNA or RNA for the presence of pre-determined polymorphisms. An appropriate test kit may include one or more of the following reagents or instruments: a means to detect the binding of the agent to the polymorphism, an enzyme able to act on a polynucleotide (typically a polymerase or restriction enzyme), suitable buffers for enzyme reagents, PCR primers which bind to regions flanking the polymorphism, a positive or negative control (or both), a gel electrophoresis apparatus and a means to isolate DNA from a sample. The product may utilise one of the chip technologies as described by the current state of the art. The test kit would include printed or machine readable instructions setting forth the correlation between the presence of a specific polymorphism or genotype and the likelihood that a subject with IBS will respond favorably to therapy with a 5HT ligand.

a biochemical test which analyses materials derived from the subject's body, including proteins or metabolites, that indicate the presence of a pre-determined polymorphism. An appropriate test kit would comprise a molecule, aptamer, peptide or antibody (including an antibody fragment) that specifically binds to a predetermined polymorphic region (or a specific region flanking the polymorphism), or a binding agent as defined herein. The product may additionally comprise one or more additional reagents or instruments (as are known in the art). The test kit would also include printed or machine-readable instructions setting forth the correlation between the presence of a specific polymorphism or genotype and the likelihood that a subject with IBS will respond favorably to therapy with a 5HT ligand.

Having such a test kit, in whatever form, facilitates the collection of phenotype data from widely diverse patient locations. Test kit results can be sent to centralized locations for genotype analysis in facilitating the identification of responder and non-responder populations.

In this Example, a method is described for screening a subject diagnosed with IBS or another gastrointestinal disorder treatable by 5HT ligands, to determine the likelihood they will respond in a particular way to treatment with a 5HT ligand, more particularly a 5HT3 antagonist, and more particularly alosetron. Subjects are mammalian, and preferably humans. The method comprises screening the subject for a polymorphism in the 5HTT gene that has previously been associated with a high or low incidence of a particular desirable therapeutic outcome (compared to the incidence in subjects with other genotypes), or associated with a high or low incidence of an undesired side effect (compared to the incidence in subjects with other genotypes), and then classifying the subject as a responder, a partial responder or a non-responder.

Treatment of a subject with a 5HT ligand comprises administration of an effective amount of the pharmaceutical agent to a subject in need thereof. The dose of agent is determined according to methods known and accepted in the pharmaceutical arts, and can be determined by those skilled in the art. A suitable dosage range and plasma concentration for alosetron are provided in the disclosure of U.S. Pat. No. 5,360,800, the entire disclosure of which is hereby incorporated herein by reference.

### Example 2

### Assay of Insertion/Deletion Polymorphism in 5HTT Gene

Genetic samples were obtained from 219 female human subjects enrolled in clinical trials of alosetron for the treatment of IBS. Using PCR technology as is known in the art, an insertion/deletion genetic marker was assayed in the 5-hydroxytryptamine transporter gene (5HTT gene). The alleles were labeled as “del” (deletion) or “ins” (insertion) resulting in three possible genotypes (del/del; del/ins or ins/ins).

The insertion/deletion marker was in the 5′ untranslated region of the 5HTT gene. The deletion polymorphism (allele “del”) had SEQ ID NO: 1; the insertion polymorphism (allele “ins”) had SEQ ID NO:2 (insertion shown in bold typeface):

| | |
|---|---|
| ggcgttgccg ctctgaatgc cagccctaac ccctaatgtc cctactgcag cctcccagca 60 | (SEQ ID NO:1) |
| tcccccctgc aacctcccag caactccctg tacccctcct aggatcgctc ctgcatcccc 120 | |
| cattatcccc cccttcactc ctcgcggcat cccccctgca ---------- ---------- 180 | |
| ---------- ---------- ----ccccca gcatcccccc tgcagccctt ccagcatccc 240 | |
| cctgcacctc tcccaggatc tcccctgcaa cccccattat cccccctgca cccctcgcag 300 | |
| tatcccccct gcacccccca gcatcccccc atgcaccccc ggcatccccc ctgcacccct 360 | |
| ccagcattct ccttgcaccc taccagtatt cccccgcatc ccggcctcca agcctcccgc 420 | |
| ccaccttgcg gtccccgccc tggcgtctag gtggcaccag aatcccgcgc ggactccacc 480 | |
| cgctgggagc tgccctcgct tgcccgtggt tgtccagctc agtc cctc 528 | |
| ggcgttgccg ctctgaatgc cagccctaac ccctaatgtc cctactgcag cctcccagca 60 | (SEQ ID NO:2) |
| tcccccctgc aacctcccag caactccctg tacccctcct aggatcgctc ctgcatcccc 120 | |
| cattatcccc cccttcactc ctcgcggcat cccccctgca ccccccagca tcccccctgc 180 | |
| agccccccca gcatctcccc tgcaccccca gcatcccccc tgcagccctt ccagcatccc 240 | |
| cctgcacctc tcccaggatc tcccctgcaa cccccattat cccccctgca cccctcgcag 300 | |
| tatcccccct gcacccccca gcatcccccc atgcaccccc ggcatccccc ctgcacccct 360 | |
| ccagcattct ccttgcaccc taccagtatt cccccgcatc ccggcctcca agcctcccgc 420 | |
| ccaccttgcg gtccccgccc tggcgtctag gtggcaccag aatcccgcgc ggactccacc 480 | |
| cgctgggagc tgccctcgct tgcccgtggt tgtccagctc agtc cctc 528 | |

The deleted segment comprised nucleotides 161-204 of SEQ ID NO:2. PCR primer sequences are in underlined typeface.

The present 5HTT genotypes were approximately evenly distributed. Of the 219 subjects genotyped for the 5HTT marker, 71(32.4%) were del/del 5HTT, 75 (34.2%) were del/ins 5HTT and 73 (33.3%) were ins/ins 5HTT.

The “del” allele represents a deletion of approximately 44 base pairs in the 5′ untranslated region of the 5HTT gene. The del/del genotype results in a lower transcription efficiency, lower production of 5HTT, and reduced basal 5HT re-uptake (compared to the del/ins or ins/ins genotype).

### Example 3

### Correlation of Genotype and Phenotype

The subjects' response to alosetron in the clinical trial setting was reviewed and correlated with genotype. In the double-blind, placebo controlled clinical trials, subjects received 12 weeks of treatment with either alosetron or a placebo. A favorable response to alosetron was when a subject reported relief of IBS symptoms during six weeks of the twelve week trial. The incidence of various other effects, including constipation, was also recorded.

The response of subjects to treatment with alosetron in the clinical trial was stratified according to genotype.

In each of the three 5HTT genotypes alosetron was more effective than placebo in producing relief (FIG. 1). However, in the del/del genotype group (homozygous for the deletion polymorphism), an increased incidence of relief of IBS symptoms was seen (increased compared to other 5HTT genotypes). (FIG. 1). Relief of IBS symptoms with alosetron was achieved in 68% of del/del subjects (21/31); 64% of del/ins subjects (21/33); and 58% of ins/ins subjects (22/38).

The occurrence of constipation during alosetron treatment in the clinical trial was stratified according to genotype. Alosetron treated subjects with the del/del genotype showed a reduced incidence of constipation compared to the del/ins and ins/ins 5HTT genotype groups (FIG. 2) Constipation was reported in 21% of the total group of subjects receiving alosetron (n-102). In del/del subjects (n=31), 4 (13%) reported constipation; in del/ins subjects (n=33), 10 (30%) reported constipation; and in ins/ins subjects (n=38), 8 (21)% reported constipation. FIG. 2.

Subjects with the del/del 5HTT genotype showed an increased incidence of favourable therapeutic response, with higher incidence of relief of IBS symptoms and lower incidence of constipation, when compared with subjects with del/ins and ins/ins 5HTT genotypes (FIG. 3). The del/del 5HTT genotype can thus be considered as a responder group, leaving the del/ins and ins/ins 5HtTT genotype groups being considered as qualified responders or non-responders.

### Example 4

### Genotyping of Individuals for 5HTT Polymorphisms

DNA samples are obtained from a population of subjects with gastrointestinal disease, and genomic DNA is extracted using standard procedures (automated extraction or using kit formats). The genotypes of the subjects, and any control individuals utilized, are determined for polymorphisms within the 5HTT gene sequence, using either PCR, PCR-RFLP, Taqman alelic discrimination assays, or any other suitable technique as is known in the art.

If a specific polymorphism resides in an amplification product that is of sufficient physical size (e.g., an insertion/deletion polymorphism of multiple bases), a simple size discrimination assay can be employed to determine the genotype of an individual. In this case, two primers are employed to specifically amplify the gene of interest in a region surrounding the site of the polymorphism. PCR amplification is carried out, generating products which differ in length, dependent on the genotype (insertion or deletion) they possess. When subjected to gel electrophoresis, the differently sized products are separated, visualized, and the specific genotypes interpreted directly.

PCR-RFLP (polymerase chain reaction-restriction fragment length polymorphism) assays may also be utilized as is known in the art to detect polymorphisms. For each polymorphic site, a PCR-RFLP assay employs two gene-specific primers to anneal to, and specifically amplify a segment of genomic DNA surrounding the polymorphic site of interest. Following PCR amplification, specific restriction endonuclease enzymes are employed to digest the PCR products produced. The enzyme utilized for an assay is selected due to its specific recognition sequence which it requires to bind to, and cleave the PCR product in the presence/absence of the polymorphism, yielding fragments diagnostic of the specific base present at the polymorphic site. Following cleavage by the restriction enzyme, gel electrophoresis is employed to separate and visualize the fragments produced.

Taqman assays, as are known in the art, may also be utilized to identify polymorphisms. For each polymorphic site the allelic discrimination assay uses two allele specific probes labeled with a different fluorescent dye at their 5′ ends but with a common quenching agent at their 3′ ends. Both probes have a 3′ phosphate group so that Taq polymerase cannot add nucleotides to them. The allele specific probes comprising the sequence encompassing the polymorphic site and will differ only in the sequence at this site (this is not necessarily true, the allele-specific probes can be shifted relative to each other such that they are not identical in length or composition. However, where they cover the same DNA region they are identical apart from the polymorphic site of interest). The allele specific probes are only capable of hybridizing without mismatches to the appropriate site.

The allele specific probes are used in conjunction with two primers, one of which hybridizes to the template 5′ of the two specific probes, whilst the other hybridizes to the template 3′ of the two probes. If the allele corresponding to one of the specific probes is present, the specific probe will hybridize perfectly to the template. The Taq polymerase, extending the 5′ primer, will then remove the nucleotides from the specific probe, releasing both the fluorescent dye and the quenching agent. This will result in an increase in the fluorescence from the dye no longer in close proximity to the quenching agent.

If the allele specific probe hybridizes to the other allele the mismatch at the polymorphic site will inhibit the 5′ to 3′ endonuclease activity of Taq and hence prevent release of the fluorescent dye.

The ABI7700 sequence detection system is used to measure the increase in the fluorescence from each specific dye at the end of the thermal cycling PCR directly in PCR reaction tubes. The information from the reactions is then analyzed. If an individual is homozygous for a particular allele only fluorescence corresponding to the dye from that specific probe will be released, but if the individual is heterozygous, then both dyes will fluoresce.

The genotypes of the individuals are then correlated with their phenotypic response to treatment with a 5HT ligand. Responses that vary among the genetic subpopulations are identified as either responders, partial responders or non-responders. In the method of the present invention, once the non-responder population has been identified, it is assumed that a different genotype is present in that population, which is expressing one or more different proteins that comprise a different biochemical pathway that is the underlying cause of the disease as it is seen in the clinic. Hence the non-responder population becomes the focus of a subsequent clinical trial, in which a drug candidate is administered that has been shown to interact with one or more targets thought to be part of the disease pathway in this population that did not respond to the drug administered in the first trial. If the second trial demonstrates that the second drug candidate elicits a favorable response in the entire population that did not respond to the drug candidate in the first drug trial, then it is apparent that the entire population of patients that started the trials in the first place are now the beneficiaries of safe and effective drug treatments for that clinical definition of disease. It is believed that in many cases, there will be more than two iterations of such clinical trials, reflecting that there are a like number of alternative genotypes that manifest that clinical definition of disease. For example, there may be as many as six distinct genotypes that manifest the disease classified as non-insulin dependant diabetes mellitus. As such, any number of iterations of clinical trials can be run, centered around the method of the invention, that is, that in any given iteration that produces a population on non-responders, the population of non-responders represents a whole new group of patients that likely have a different genotype that is treatable by a drug that is different from the drug tested in the previous iteration of clinical trials.

What is claimed:

1. A method of conducting clinical drug trials by pharmacogenetic stratification of a patient population, comprising the steps of:
(a) Conducting a first clinical drug trial on a patient population, such that said drug trial identifies an association between a phenotype and a genotype;
(b) Separating said patient population in said clinical drug trial into subpopulations of responders and non-responders;
(c) Conducting a subsequent clinical drug trial on a non-responder patient population such that said subsequent drug trial identifies a subsequent association between a phenotype and a genotype;
(d) Separating the patient population of step (c) into subsequent responder and subsequent non-responder patient populations; and
(e) Repeating steps (c) and (d) through as many iterations as desired.

2. A method of conducting clinical drug trials by pharmacogenetic stratification of a patient population, comprising the steps of:
(a) Conducting a first clinical drug trial on a patient population, such that said drug trial identifies an association between a phenotype and a genotype, where said phenotype is an observed response or lack thereof to a drug being studied in said drug trial;
(b) Separating said patient population in said clinical drug trial into subpopulations of responders and non-responders;
(c) Conducting a subsequent clinical drug trial on a non-responder patient population such that said subsequent drug trial identifies a subsequent association between a phenotype and genotype, where said phenotype is an observed response or lack thereof to a drug being studied ins said subsequent drug trial;
(d) Separating the patient population of step (c) into subsequent responder and subsequent non-responder patient populations; and
(e) Repeating steps (c) and (d) through as many iterations as desired.

3. A method of conducting clinical drug trials by pharmacogenetic stratification of a patient population, comprising the steps of:
(a) Conducting a first clinical drug trial on a patient population, such that said drug trial identifies an association between a phenotype and a genotype, where said phenotype is an observed response or lack thereof to a drug being studied in said drug trial;
(b) Separating said patient population in said clinical drug trial into subpopulations of responders and non-responders;
(c) Identifying a target for drug intervention for non-responders;
(d) Screening candidate drug compounds against said target until a candidate drug is found;
(e) Conducting a subsequent clinical drug trial on a non-responder patient population such that said subsequent drug trial identifies a subsequent association between a phenotype and genotype;
(f) Separating the patient population of step (e) into subsequent responder and subsequent non-responder patient populations; and
(g) Repeating steps (c) and (f) through as many iterations as desired.

4. A method of conducting clinical drug trials by pharmacogenetic stratification of a patient population, comprising the steps of:
(a) Conducting a first clinical drug trial on a patient population, such that said drug trial identifies an association between a phenotype and a genotype,
(b) Separating said patient population in said clinical drug trial into subpopulations of responders and non-responders;
(c) Conducting a subsequent clinical drug trial on a non-responder patient population such that said subsequent drug trial identifies a subsequent association between a phenotype and a genotype;
(d) Separating the patient population of step (c) into subsequent responder and subsequent non-responder patient populations; and
(e) Repeating steps (c ) and (d) through as many iterations as desired.

5. A method of designing novel drug therapies by pharmacogenetic stratification of a patient population, comprising the steps of:
(a) Conducting a first clinical drug trial on a patient population, such that said drug trial identifies an association between a phenotype and a genotype;
(b) Separating said patient population in said clinical drug trial into subpopulations of responders and non-responders;
(c) Conducting a subsequent clinical drug trial on a non-responder patient population such that said subsequent drug trial identifies a subsequent association between a phenotype and a genotype;
(d) Separating the patient population of step (c) into subsequent responder and subsequent non-responder patient populations; and
(e) Repeating steps (c)and (d) through as many iterations as desired.

6. A method of developing discrete drugs for different patients who have clinically similar disease phenotypes, but who have discrete genotypes, by pharmacogenetic stratification of a patient population, comprising the steps of:
(a) Conducting a first clinical drug trial on a patient population, such that said drug trial identifies an association between a phenotype and a genotype;
(b) Separating said patient population in said clinical drug trial into subpopulations of responders and non-responders;
(c) Conducting a subsequent clinical drug trial on a non-responder patient population such that said subsequent drug trial identifies a subsequent association between a phenotype and a genotype;
(d) Separating the patient population of step (c) into subsequent responder and subsequent non-responder patient populations; and
(e) Repeating steps (c)and (d) through as many iterations as desired

7. A method of developing a drug for different patients who have like genotypes but that present clinical symptoms that are categorized into different diagnostic criteria, by pharmacogenetic stratification of a patient population, comprising the steps of:
(a) Conducting a first clinical drug trial on a patient population, such that said drug trial identifies an association between a phenotype and a genotype;
(b) Separating said patient population in said clinical drug trial into subpopulations of responders and non-responders;
(c) Conducting a subsequent clinical drug trial on a non-responder patient population such that said subsequent drug trial identifies a subsequent association between a phenotype and a genotype;
(d) Separating the patient population of step (c) into subsequent responder and subsequent non-responder patient populations; and
(e) Repeating steps (c)and (d) through as many iterations as desired.

8. The method of claim 1, where said phenotype is a diagnosis of Alzheimer's Disease.

9. The method of claim 1, where said phenotype is a diagnosis of irritable bowel syndrome.

10. The method of claim 1, where said phenotype is a diagnosis of migraine headache.

11. The method of claim 1, where said phenotype is a diagnosis of psoriasis.

12. The method of claim 1 where said phenotype is a diagnosis of non-insulin dependant diabetes mellitus.

13. The method of claim 1 where said phenotype is a diagnosis of asthma.

14. The method claim 1 where said phenotype is a diagnosis of chronic obstructive pulmonary disease.

15. The method of claim 1, where said phenotype is a diagnosis of bipolar disorder.

16. The method of claim 1, where said phenotype is a diagnosis of depression.

17. The method of claim 1, where said phenotype is a diagnosis of one or more forms of epilepsy.

18. The method as claimed in claim 1, in which the population of a first or subsequent clinical drug trial can alternatively be separated into responder, non-responder and partial-responder populations.

19. The method as claimed in claim 1, and further wherein any nucleotide sequence data, amino acid sequence data, protein-protein interaction data, clinical diagnosis data or statistics data generated by the above procedure is stored in electronically readable media and is communicated via telecommunication means between at least two electronic computing devices.
